(19) United States
(12) Patent Application Publication
(10) Pub. No.: US 2024/0245882 A1
(43) Pub. Date: Jul. 25, 2024

(54) SIGNAL PROCESSING DEVICE, SIGNAL PROCESSING METHOD, AND PROGRAM

(71) Applicant: NIPPON TELEGRAPH AND TELEPHONE CORPORATION, Tokyo (JP)

(72) Inventors: Hiroaki ITO, Tokyo (JP); Hironobu CHIBA, Tokyo (JP); Tatsuya KAKO, Tokyo (JP); Kenichi NOGUCHI, Tokyo (JP)

(73) Assignee: NIPPON TELEGRAPH AND TELEPHONE CORPORATION, Tokyo (JP)

(21) Appl. No.: 18/681,017

(22) PCT Filed: Aug. 4, 2021
(86) PCT No.: PCT/JP2021/028973

§ 371 Date: Feb. 2, 2024

(51) Int. Cl. A61M 21/02; A61M 21/00; H04S 1/00; H04S 7/00

(52) U.S. Cl. CPC A61M 21/02; H04S 1/007; H04S 7/307; A61M 2021/0027; H04S 2400/15

(57) ABSTRACT

The signal processing device includes: a beat sound frequency determination unit that determines an optimal beat sound frequency of a user on the basis of a brain wave signal of the user; and a binaural beat signal generation unit that acquires an ambient environment sound, converts a sample acoustic signal prepared in advance into a binaural beat, on the basis of an optimal beat sound frequency, and mixes the binaural-beat-converted sample acoustic signal with the ambient environment sound, to generate a binaural-beat-converted ambient environmental sound signal.

### TECHNICAL FIELD

The present invention relates to a signal processing device, a signal processing method, and a program that receive an ambient environmental sound and output an environmental sound optimized to synchronize a desired brain wave band.

### BACKGROUND ART

A technique has been proposed in which a certain specific brain wave is induced by intervention of a user by various means and effects (concentration, relaxation, etc.) induced in specific brain wave synchronization are promoted. There is a technique that presents binaural beats (hereinafter abbreviated as BB) based on pure tones in order to induce certain brain waves.

Binaural beats are two pure tones of slightly different frequencies presented to the two ears, producing a beat sound (hum sound) that corresponds to the frequency difference. NPL 1 discloses that various effects are realized when the brain waves are synchronized with beat sounds (hum sounds).

### CITATION LIST

### Non Patent Literature

[NPL 1] Ross, B., Lopez, M. D. “40-Hz Binaural beats enhance training to mitigate the attentional blink.” Sci Rep 10, 7002 (2020)

### SUMMARY OF INVENTION

### Technical Problem

It is known that various effects are generated when brain waves are synchronized with binaural beats. Although it is necessary to determine the frequency of the binaural beat in order to achieve a desired effect, it is assumed that depending on the individual differences of users, there are differences in the effect even if binaural beats of the same frequency are listened to.

Thus, an object of the present invention is to provide a signal processing device capable of generating a binaural beat signal in consideration of individual user differences.

### Solution to Problem

A signal processing device of the present invention includes a beat sound frequency determination unit and a binaural beat signal generation unit.

The beat sound frequency determination unit determines an optimal beat sound frequency of a user on the basis of a brain wave signal of the user. The binaural beat signal generation unit acquires an ambient environment sound, converts a sample acoustic signal prepared in advance into a binaural beat, on the basis of an optimal beat sound frequency, and mixes the binaural-beat-converted sample acoustic signal with the ambient environment sound, to generate a binaural-beat-converted ambient environmental sound signal.

### Advantageous Effects of Invention

According to the signal processing device of the present invention, a binaural beat signal can be generated in consideration of individual user differences.

### BRIEF DESCRIPTION OF DRAWINGS

FIG. 1 is a block diagram showing a configuration of a signal processing device according to Example 1.

FIG. 2 is a flowchart showing an operation of the signal processing device according to Example 1.

FIG. 3 is a diagram showing a comparative example of a brain wave power in a rest state and a brain wave power in a BB sound listening state.

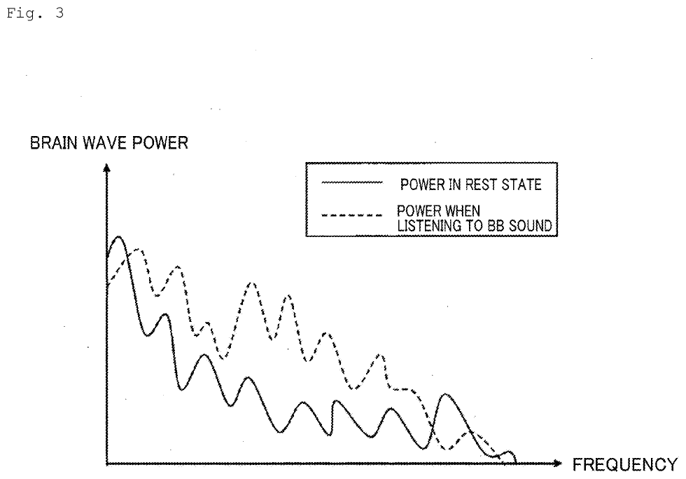

FIG. 4 is a diagram for explaining the relationship between a power ratio and an optimal beat frequency.

FIG. 5 is a diagram showing a detailed functional configuration example of a binaural beat signal generation unit of the signal processing device of Example 1.

FIG. 6 is a block diagram showing a configuration of a signal processing device according to Example 2.

FIG. 7 is a flowchart showing an operation of the signal processing device according to Example 2.

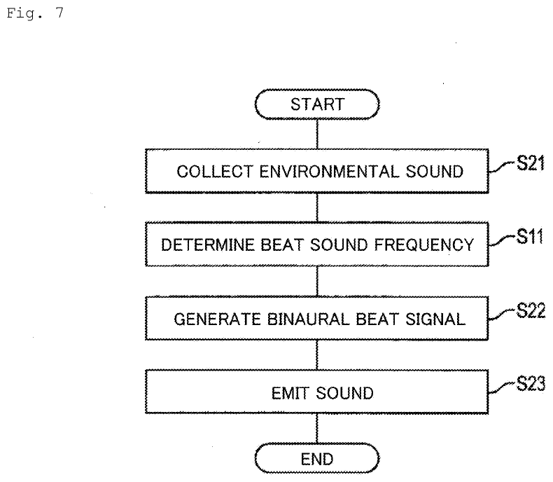

FIG. 8 is a diagram showing a functional configuration example of a computer.

### DESCRIPTION OF EMBODIMENTS

The following describes an embodiment of the present invention in detail. Note that components having the same function are denoted by the same number and redundant description thereof is omitted.

### Example 1

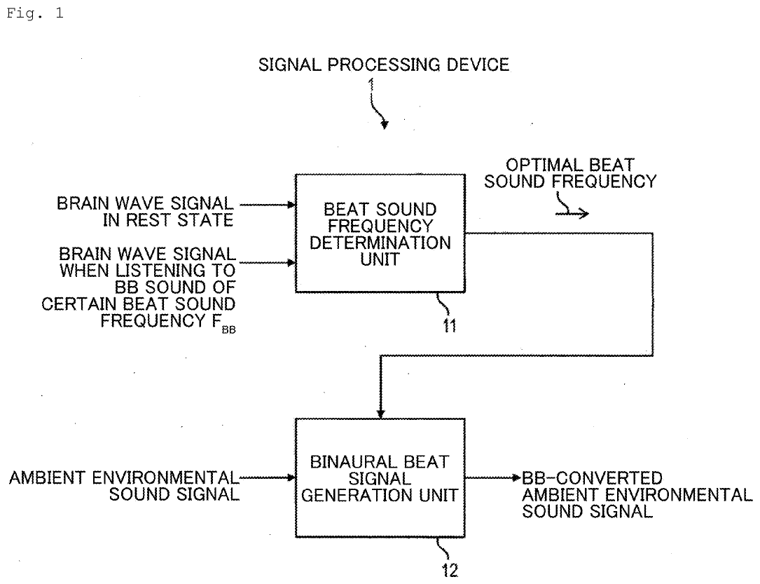

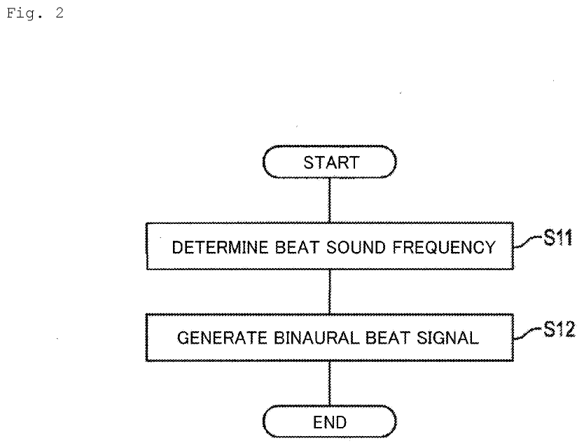

As shown in FIG. 1, a signal processing device 1 of Example 1 includes a beat sound frequency determination unit 11 and a binaural beat signal generation unit 12. Operations of the respective functional configurations will be described hereinafter with reference to FIG. 2.

### <Beat Sound Frequency Determination Unit 11>

Input: A brain wave signal of the user in a rest state, a brain wave signal of the user when listening to a BB sound of a certain beat sound frequency f_{BB}

Output: An optimal beat sound frequency of the user

Processing: The beat sound frequency determination unit 11 determines an optimal beat sound frequency of the user on the basis of a ratio between a power spectrum in a specific frequency band of the brain wave signal of the user in a rest state and a power spectrum in a specific frequency band of the brain wave signal of the user listening to a binaural beat sound of the certain beat sound frequency f_{BB} (S11). This processing is an example, and any means may be used as long as the input brain wave signal of the user can be modulated into an optimal brain wave signal. For example, environmental noise described hereinafter may be obtained by converting a desired beat into a binaural beat, or a natural sound may be used instead of the environmental noise. It is preferred that acoustic signals that do not bore the user be converted into binaural beats. The term “optimal” described in step S11 may be set according to the purpose of brain waves when the user is concentrating, not sleepy, and learning efficiently.

### <<Details of S11>>

The beat sound frequency determination unit 11 calculates a power spectrum from the brain wave signal of the user in a rest state and the brain wave signal of the user listening to the BB sound, and calculates a power ratio in a specific frequency band. It is assumed that the specific frequency band corresponds to a brain wave to be induced and is set in advance.

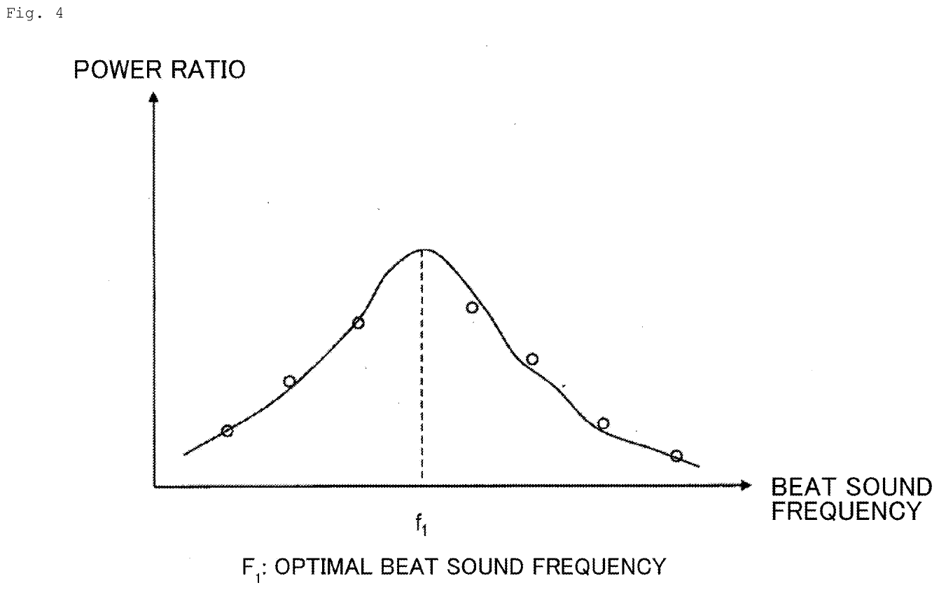

The beat sound frequency determination unit 11 repeats the processing described above while changing the frequency of the beat sound, and measures the relationship between the frequency of the beat sound and the power ratio (see the example show in FIG. 4). It is assumed that the range to be changed is set in advance near the brain wave band to be induced.

The beat sound frequency determination unit 11 determines the optimal beat sound frequency by using a means such as regression, from the relationship between the beat sound frequency and the power ratio (refer to the example shown in FIG. 4. The frequency at which the power ratio is the maximum value is set as the optimal beat sound frequency).

### <Binaural Beat Signal Generation Unit 12>

Input: An ambient environmental sound signal and an optimal beat sound frequency of the user

Output: An ambient environmental sound signal subjected to binaural beat conversion based on the optimal beat sound frequency of the user

Processing: The binaural beat signal generation unit 12 acquires an ambient environment sound, converts a sample noise prepared in advance into a binaural beat on the basis of the optimal beat sound frequency, and mixes the binaural-beat-converted sample noise with the ambient environment sound, to generate a binaural-beat-converted ambient environmental sound signal (S12). In S12, an environmental sound or a natural sound can be used instead of the sample noise.

### <<Details of S12>>

The binaural beat signal generation unit 12 BB-converts a sample noise (e.g., pink noise or white noise) held in the device based on the optimal beat sound frequency of the user. As for the BB-conversion means, any method may be employed as long as the beat sound of a beat sound frequency optimal for an individual is generated by the left and right signals. For example, there is a method such as pitch shifting. It is sufficient if a pair of acoustic signals can be generated that have the same properties except for a desired frequency shift.

It is preferred that sound emission be performed in such a manner that one signal out of two types of acoustic signals for generating a binaural beat reaches the right ear of the user and the other signal reaches the left ear of the user. The binaural-beat-converted signal may be monauralized and the monauralized signal may be emitted in such a manner that it reaches both ears.

The binaural beat signal generation unit 12 mixes the BB sound generated by the processing described above with the ambient environmental sound signal. The mixing ratio is set in advance.

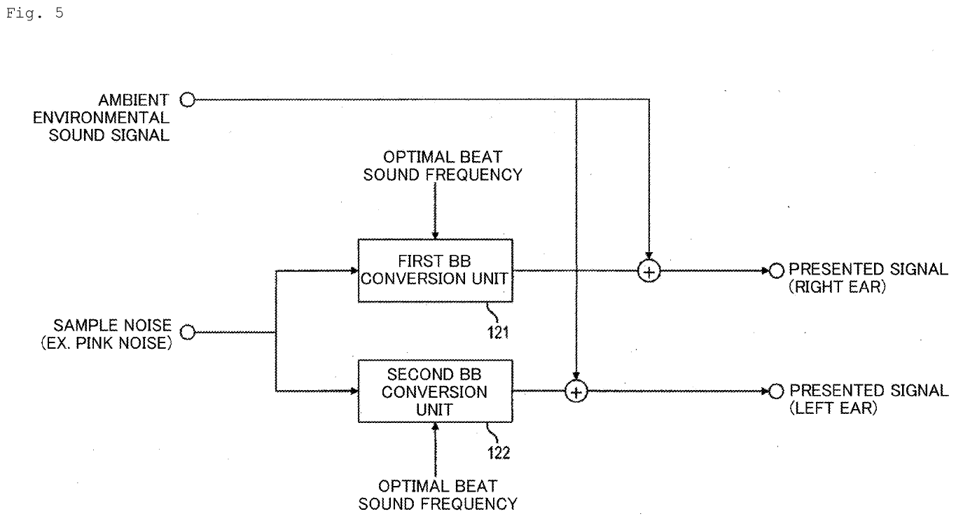

Note that, as shown in FIG. 5, the binaural beat signal generation unit 12 may generate two-channel signals by mixing two types of sample noises having different frequencies based on the optimal beat sound frequency, with the ambient environmental sound. More specifically, the binaural beat signal generation unit 12 includes a first BB conversion unit 121 and a second BB conversion unit 122. The first BB conversion unit 121 may generate a signal of a first channel by mixing a sample noise whose frequency is made different on the basis of an optimal beat sound frequency and an ambient environmental sound, and the second BB conversion unit 122 may generate a signal of a second channel by mixing a sample noise whose frequency is made different on the basis of an optimal beat sound frequency and an ambient environmental sound.

### <Effects>

According to the signal processing device 1 of the present example, since the current brain wave state of the individual user is grasped to determine the frequency of the beat sound, the effect of synchronizing the target brain wave band is high. Further, since the pitch of the ambient environmental sound itself is not shifted, but the noise signals converted into binaural beats are mixed, the user does not feel uncomfortable.

### Example 2

Next, a signal processing device of Example 2, which converts ambient environmental sounds around a user into binaural beats, will be described. In the above-described embodiment, the user is allowed to listen to a signal obtained by mixing binaural-beat-converted noise and ambient environmental sound to synchronize the desired frequency band of the brain wave of the user, but in the present embodiment, the ambient environmental sound itself of the user is converted into a binaural beat for the user to listen to.

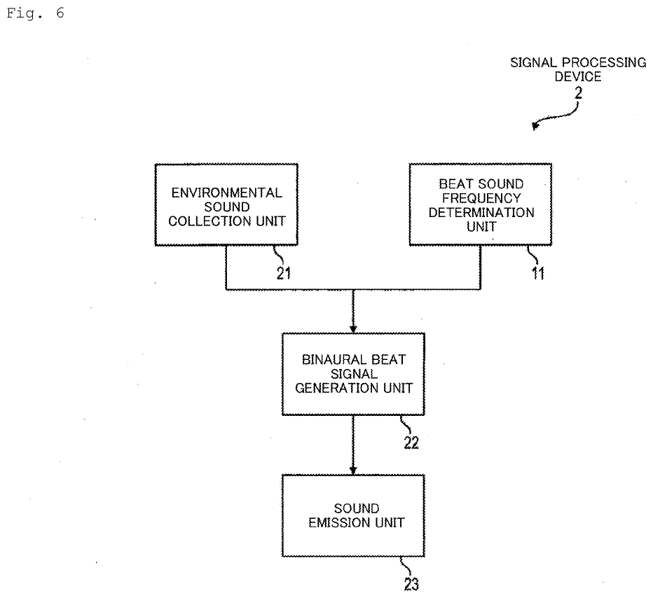

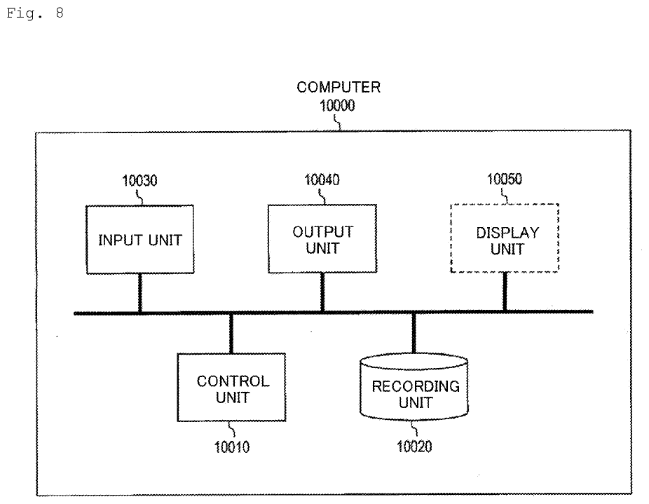

The functional configuration of the signal processing device 2 according to the present example will be described below with reference to FIG. 6. As shown in FIG. 6, the signal processing device 2 of the present example includes an environmental sound collection unit 21, the beat sound frequency determination unit 11, the binaural beat signal generation unit 22, and a sound emission unit 23. Operations of the respective functional configurations will be described hereinafter with reference to FIG. 8.

### <Environmental Sound Collection Unit 21>

Input: Ambient environmental sound reaching user

Output: Collected environmental sound

Processing: The environmental sound collection unit 21 collects the ambient environmental sound reaching the user, to acquire the collected sound environmental sound (S21).

### <Beat Sound Frequency Determination Unit 11>

Input: A brain wave signal of the user in a rest state, a brain wave signal of the user when listening to a BB sound of a certain beat sound frequency f_{BB}

Output: An optimal beat sound frequency of the user

Processing: The processing of the beat sound frequency determination unit 11 is the same as that described in Example 1. That is, the beat sound frequency determination unit 11 associates the brain wave of the user with the binaural beat, and calculates an optimal beat sound frequency for achieving a desired effect (S11).

### <Binaural Beat Signal Generation Unit 22>

Input Collected environmental sound

Output: Collected environmental sound converted into a binaural beat based on an optimal beat sound frequency of the user

Processing: The binaural beat signal generation unit 22 converts the collected environmental sound into a binaural beat on the basis of the optimal beat sound frequency of the user (S22). In the binaural beat signal generation unit, the noise prepared in advance is converted into a binaural beat, but this is different in that the collected environmental sound is converted into a binaural beat instead of the noise.

### <Sound Emitting Unit 23>

Input: Collected environmental sound converted into a binaural beat based on an optimal beat sound frequency of the user

Output: Sound

Processing: The sound emission unit 23 emits, to the user, the collected environmental sound converted into a binaural beat (S23). The sound emission unit 23 may emit sound in such a manner that one of two signals converted into binaural beats, i.e., having different frequencies, reaches the left ear, and the other signal reaches the right ear, or in such a manner that two signals with different in frequencies are monauralized, which then reach the left and right ears.

In this case, a configuration is possible in which the environmental sound that directly reaches the user and the environmental sound converted into a binaural beat arrive at a time difference that does not make the user feel uncomfortable. A configuration is also possible in which the ambient environmental sound does not directly reach the user. For example, active noise cancellation may be performed, or the user may be placed in a space where external sound hardly arrives. When the user is placed in a space where external sound hardly arrives, environmental noise arriving outside the space may be converted into a binaural beat as an ambient environmental sound.

### <Additional Note>

The device according to the present invention includes, as a single hardware entity, for example, an input unit to which a keyboard or the like can be connected, an output unit to which a liquid crystal display or the like can be connected, a communication unit to which a communication device (for example, a communication cable) capable of communicating with the outside of the hardware entity can be connected, a CPU (Central Processing Unit which may also include a cache memory and a register), a RAM or ROM serving as a memory, an external storage device which is a hard disk, and a bus that connects the input unit, the output unit, the communication unit, the CPU, the RAM, the ROM, and the external storage device such that data can be exchanged therebetween. As necessary, a device (drive) capable of reading and writing a storage medium such as a CD-ROM may be provided in the hardware entity. A general-purpose computer or the like is an example of a physical entity including such hardware resources.

A programs required to implement the above-described functions, data required to process the program, and the like are stored in the external storage device of the hardware entity (the program may be read and stored not only the external storage device but also, for example, in a ROM which is a ready-only storage device). Further, data or the like obtained by processing the program is appropriately stored in the RAM, the external storage device, or the like.

In the hardware entity, each program stored in the external storage device (or the ROM or the like) and the data necessary to process each program are loaded to the memory, as necessary, and are interpreted, executed, and processed by the CPU as appropriate. As a result, the CPU implements predetermined functions (configuration requirements represented as units, means, and the like as described above).

The present invention is not limited to the above-described embodiments, and appropriate changes can be made without departing from the spirit of the present invention. Further, the processes described in the embodiments are not only executed in time series in the described order, but also may be executed in parallel or individually according to a processing capability of a device that executes the processes or as necessary.

As described above, when a processing function in the hardware entity (the device according to the present invention) described in the above-described embodiments is implemented by a computer, processing content of the function to be included in the hardware entity is described by the program. By executing this program on the computer, the processing function in the above-described hardware entity is implemented on the computer.

The above-described various types of processing can be performed by reading a program executing each step of the foregoing methods to a storage unit 10020 of a computer 10000 illustrated in FIG. 8 and operating a control unit 10010, an input unit 10030, an output unit 10040, or the like.

The program describing the processing content can be recorded on a computer readable recording medium. Examples of the computer-readable recording medium include a magnetic recording device, an optical disc, a magneto-optical recording medium, and a semiconductor memory. Specifically, for example, a hard disk device, a flexible disk, a magnetic tape, or the like can be used as a magnetic recording device, a DVD (digital versatile disc), a DVD-RAM (Random Access Memory), a CD-ROM (Compact Disc Read Only Memory), a CD-R (Recordable)/RW (Rewritable), or the like can be used as an optical disc, a MO (Magneto-Optical disc) or the like can be used as a magneto-optical recording medium, and an EEP-ROM (Electronically Erasable and Programmable-Read Only Memory) or the like can be used as a semiconductor memory.

The program is distributed, for example, by sales, transfer, or lending of a portable recording medium such as a DVD or a CD-ROM on which the program is recorded. In addition, the distribution of the program may be performed by storing the program in advance in a storage device of a server computer and transferring the program from the server computer to another computer via a network.

A computer executing such a program is configured, for example, to first store a program recorded on a portable recording medium or a program transferred from a server computer temporarily in an own storage device. Then, when the processing is performed, the computer reads the program stored in the own recording medium and performs the processing according to the read program. As another execution form of the program, the computer may directly read the program from the portable recording medium and execute processing according to the program. Whenever the program is transferred from the server computer to the computer, processing according to the received program may be executed in sequence. By a so-called application service provider (ASP) type service which does not transfer a program from the server computer to the computer and implements a processing function only in response to the execution instruction and the result acquisition, the above-described processing may be executed. It is assumed that the program according to the present embodiment includes data which is information provided for processing by an electronic computer and equivalent to the program (data or the like which is not a direct command to the computer but has a property for specifying the processing of the computer).

Further, although the hardware entity is configured by a predetermined program being executed on the computer in the present embodiment, at least a part of the processing content of the hardware entity may be realized in hardware.

1. A signal processing device comprising a processor configured to execute operations comprising:
determining an optimal beat sound frequency of a user on the basis of a brain wave signal of the user;
acquiring an ambient environment sound,
converting a sample acoustic signal prepared in advance into a binaural beat, on the basis of an optimal beat sound frequency;
mixing the binaural-beat-converted sample acoustic signal with the ambient environment sound; and
generating a binaural-beat-converted ambient environmental sound signal.

2. The signal processing device according to claim 1, wherein
the generating further comprises generating two-channel signals by mixing two types of sample noises having different frequencies based on the optimal beat sound frequency, with the ambient environment sound.

3. The signal processing device according to claim 1, wherein
the generating further comprises generating a signal of a first channel by mixing a sample noise whose frequency is made different on the basis of the optimal beat sound frequency and the ambient environment sound, and a signal of a second channel by mixing a sample noise whose frequency is made different on the basis of the optimal beat sound frequency and the ambient environment sound.

4. A signal processing device comprising a processor configured to execute operations comprising:
determining an optimal beat sound frequency of a user on the basis of a brain wave signal of the user;
acquiring an ambient environment sound of the user; and
converting the acquired ambient environment sound into a binaural beat on the basis of the optimal beat sound frequency.

5. A signal processing method, comprising:
determining an optimal beat sound frequency of a user based on a brain wave signal of the user;
acquiring an ambient environment sound, converting a sample acoustic signal prepared in advance into a binaural beat based on the optimal beat sound frequency;
mixing the binaural-beat-converted sample acoustic signal with the ambient environment sound; and
generating a binaural-beat-converted ambient environmental sound signal.

6-7. (canceled)

8. The signal processing device according to claim 1, wherein the brain wave signal represents a signal of the user in a rest state.

9. The signal processing device according to claim 1, wherein the determining an optimal beat sound frequency of the user is based on a combination of a first brain wave signal of the user in a rest state and a second brain wave of the user while listening to a binaural beat sound at a beat sound frequency.

10. The signal processing device according to claim 1, wherein the determining the optimal beat sound frequency of a user further comprises
changing a frequency of a binaural beat sound within a range of a brain wave band of the brain wave signal of the user to be induced.

11. The signal processing device according to claim 1, wherein the sample acoustic signal includes a white noise.

12. The signal processing device according to claim 4, wherein the brain wave signal represents a signal of the user in a rest state.

13. The signal processing device according to claim 4, wherein the determining an optimal beat sound frequency of the user is based on a combination of a first brain wave signal of the user in a rest state and a second brain wave of the user while listening to a binaural beat sound at a beat sound frequency.

14. The signal processing device according to claim 4, wherein the determining the optimal beat sound frequency of a user further comprises
changing a frequency of a binaural beat sound within a range of a brain wave band of the brain wave signal of the user to be induced.

15. The signal processing method according to claim 5, wherein
the generating further comprises generating two-channel signals by mixing two types of sample noises having different frequencies based on the optimal beat sound frequency, with the ambient environment sound.

16. The signal processing method according to claim 5, wherein
the generating further comprises generating a signal of a first channel by mixing a sample noise whose frequency is made different on the basis of the optimal beat sound frequency and the ambient environment sound, and a signal of a second channel by mixing a sample noise whose frequency is made different on the basis of the optimal beat sound frequency and the ambient environment sound.

17. The signal processing method according to claim 5, wherein the brain wave signal represents a signal of the user in a rest state.

18. The signal processing method according to claim 5, wherein the determining an optimal beat sound frequency of the user is based on a combination of a first brain wave signal of the user in a rest state and a second brain wave of the user while listening to a binaural beat sound at a beat sound frequency.

19. The signal processing method according to claim 5, wherein the determining the optimal beat sound frequency of a user further comprises
changing a frequency of a binaural beat sound within a range of a brain wave band of the brain wave signal of the user to be induced.

20. The signal processing method according to claim 5, wherein the sample acoustic signal includes a white noise.
